SEQUENCE LISTING (17 sequences)

SEQ ID NO: 1 - protein, 180 aa, Homo sapiens
MQAEGRGTGGSTGDADGPGGPGIPDGPGGNAGGPGEAGATGGRAPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 2 - protein, 13 aa, Homo sapiens
PGIPDGPGGNAGG

SEQ ID NO: 3 - protein, 13 aa, Homo sapiens
GPGGPGIPDGPGG

SEQ ID NO: 4 - protein, 13 aa, Homo sapiens
ASGLNGCCRCGAR

SEQ ID NO: 5 - protein, 13 aa, Homo sapiens
GARGPESRLLEFY

SEQ ID NO: 6 - protein, 13 aa, Homo sapiens
TVSGNILTIRLTA

SEQ ID NO: 7 - protein, 13 aa, Homo sapiens
SGNILTIRLTAAD

SEQ ID NO: 8 - protein, 13 aa, Homo sapiens
SCLQQLSLLMWIT

SEQ ID NO: 9 - protein, 9 aa, Homo sapiens
SLLMWITQC

SEQ ID NO: 10 - protein, 13 aa, Homo sapiens
SLLMWITQCFLPV

SEQ ID NO: 11 - protein, 13 aa, Homo sapiens
ATGGRGPRGAGAA

SEQ ID NO: 12 - protein, 13 aa, Homo sapiens
SRLLEFYLAMPFA

SEQ ID NO: 13 - protein, 13 aa, Homo sapiens
EFYLAMPFATPME

SEQ ID NO: 14 - protein, 13 aa, Homo sapiens
LKEFTVSGNILTI

SEQ ID NO: 15 - protein, 13 aa, Homo sapiens
SLLMWITQCFLPV

SEQ ID NO: 16 - protein, 14 aa, Homo sapiens
SLLMWITQCFLPVL

SEQ ID NO: 17 - protein, 13 aa, Homo sapiens
WITQCFLPVFLAQ

(19) United States
(12) Patent Application Publication
(10) Pub. No.: US 2012/0171232 A1
(43) Pub. Date: Jul. 5, 2012

(54) IN VIVO EFFICACY OF NY-ESO-1 PLUS ADJUVANT

(75) Inventors: Jonathan Cebon, Victoria (AU); Ian Davis, Victoria (AU); Weisan Chen, Victoria (AU); Simon Green, Marburg (DE)

(73) Assignees: Ludwig Institute for Cancer Research; CSL Limited

(21) Appl. No.: 13/111,381

(22) Filed: May 19, 2011

Related U.S. Application Data
(63) Continuation of application No. 10/573,753, filed on Aug. 8, 2006.
(60) Provisional application No. 60/572,543, filed on May 18, 2004.
(60) Provisional application No. 60/507,175, filed on Sep. 30, 2003.

(51) Int. Cl. A61K 39/00; A61P 35/00

(52) U.S. Cl. 424/185.1

(57) ABSTRACT

The invention relates to the discovery that administration of NY-ESO-1 protein, in combination with a saponin based adjuvant leads to an unexpectedly strong immune response against NY-ESO-1 expressing cells. Preferably, the combination is administered intramuscularly.

### RELATED APPLICATIONS

This application is a continuation-in-part of application Ser. No. 60/572,543, filed on May 18, 2004, which is a continuation in part of application Ser. No. 60/507,175, filed Sep. 30, 2003, both of which are incorporated by reference in their entirety.

### FIELD OF THE INVENTION

This invention relates to effective methods for treatment and prophylaxis of cancer. More particularly, it relates to the treatment and prophylaxis of patients who either are affected with cancers, or are susceptible thereto. The cancers are characterized by expression of the cancer-testis antigen referred to as NY-ESO-1. The invention also provides information on new, CD4⁺ T cell epitopes, which bind to MHC-Class II molecules.

### BACKGROUND AND PRIOR ART

The work reported in the parent and grandparent applications is published by Marakovsky, et al., Clin. Canc. Res., 10:2879-2890 (Apr. 15, 2004); Q. Chen, et al., Proc. Natl. Acad. Sci. USA, 101(25):9363-9368 (Jun. 22, 2004), and Davis, et al., Proc. Natl. Acad. Sci. USA, 101(29):10697-10702(7/20/04). The NY-ESO-1 molecule, (SEQ ID NO: 1 herein) described in, e.g., U.S. Pat. Nos. 5,804,381; 6,274,145; 6,252,052; and 6,525,177, all of which are incorporated by reference, is particularly attractive as a potential cancer therapeutic agent, for several reasons. It is expressed widely in malignancies, including melanoma, hepatocellular carcinoma, soft tissue sarcoma, and cancers of the lung, bladder, head and neck, and breast. See Chen, et al., Proc. Natl. Acad. Sci. USA, 94:1914-1918 (1997); and Jungbluth, et al., Int. J. Canc., 92:856-860 (2001), both of which are incorporated by reference. Further, the only normal tissue type which expresses the antigen is testis tissue, as verified by both RT-PCR, and immunohistochemistry. For these reasons, it is referred to as a “cancer-testis” antigen. For a review of these, see Scanlan, et al., Cancer Immunity, 4:1(2004), incorporated by reference.

Patients with cancer who express NY-ESO-1 in their malignancies have been shown to develop spontaneous humoral and cellular CD8⁺ and CD4⁺ T cell responses against NY-ESO-1. See Stockert, et al., J. Exp. Med., 187:1349-54(1998); Jager, et al., J. Exp. Med., 187:265-270 (1998); and Jager, et al., Int. J. Cancer, 84:506-510 (1999). Further, many reports define HLA Class I or Class II restricted peptides, with amino acid sequences found in NY-ESO-1 representing CD4⁺/CD8⁺ T-cell epitopes. See the two Jager papers, supra, as well as Jager, et al., Proc. Natl. Acad. Sci. USA, 97:121980-12203 (2000); Gnjatic, et al., Proc. Natl. Acad. Sci. USA, 97:10917-10922 (2000); Jager, et al., Cancer Immunity, 2:12-24 (2002); Zeng, et al., J. Immunol., 165:1153-1159 (2000); Zarour, et al., Cancer Res., 60:4946-4952 (2000); Zarour, et al., Cancer Res., 62:213-218 (2002); Jager, et al., J. Exp. Med., 191:625-630 (2000); Zeng, et al., Proc. Natl. Acad. Sci. USA, 98:3964-3969 (2001); Gnjatic, et al., Proc. Natl. Acad. Sci. USA, 100:8862-8867 (2003); and Wang, et al., J. Immunol., 161:3598-3606 (1998). All of these references are incorporated by references in their entirety. Representative of the patent literature in this area are the patents described supra, as well as, U.S. Pat. Nos. 6,417,165 and 6,605,711 incorporated by reference. Generally, the identification of these CD4⁺ and CD8⁺ epitopes has resulted from study of patients who have developed spontaneous immune responses to the antigens expressed by their cancers.

The spontaneous or natural immunogenicity of NY-ESO-1 makes it a good potential candidate for cancer vaccination. The result of clinical trials using HLA-A2 restricted, NY-ESO-1 peptide, in combination with different adjuvants, have shown that these peptide vaccines are safe, and T cell responses can be generated in response to synthetic peptides. See Jager, et al., Proc. Natl. Acad. Sci. USA, 97:12198-12203 (2000); and Davis, et al., Proc. Am. Assoc. Cancer Res., 2774 (2002). These trials, while limited by their restriction to HLA-A2 positive patients, did nonetheless show some clinical benefit, in response to a single, HLA-A2 restricted epitope. See Jager, et al., supra.

Vaccination with full length NY-ESO-1 protein, on the other hand, arguably represents a more physiological vaccine composition, i.e., an immunogenic composition, allowing antigen uptake and processing by professional “antigen presenting cells,” or “APC's”, and cross-presentation of antigenic peptides by HLA Class I and II together with cognate helper activity. See Ploegh, Science, 304:1262-1263(2004). This has the potential to induce a broader immune response against multiple CD8⁺ and CD4⁺ T cell epitopes contained within the NY-ESO-1 sequence, as well as antibody responses. There is no need, in such cases, to limit the vaccination to HLA-A2 positive patients, thus enabling application of the vaccine to any cancer patient with a tumor that expresses NY-ESO-1.

Proteins and peptides, when formulated for use as vaccines, are preferably combined with adjuvants. The optimal adjuvant or adjuvants for use in cancer vaccines, has not been identified. An adjuvant known as ISCOM, which is one of several saponin based, adjuvants, including but not being limited to, QS-21 and variants thereof, has been shown to be safe, well tolerated, and able to induce strong antibody and T cell responses, in animals and humans. ISCOM is described in, e.g., U.S. Pat. No. 6,352697, and PCT application WO96/11711, both of which are incorporated by reference. In essence, an ISCOM vaccine describes a vaccine comprising saponin, sterol and antigen wherein the antigen is associated with the saponin:sterol complex via hydrophobic interaction. An ISCOMATRIX vaccine comprises the same components but the antigen is not associated by hydrophobic interactions. Also see Barr, et al., Immunol. Cell Biol., 74:8-25 (1996); and Ennis, et al., Virology, 259:256-261 (1999). Although these reports do not refer to cancer vaccines specifically the results that have been reported for ISCOM make it an attractive adjuvant for cancer vaccination.

The parent applications, referred to supra, described how vaccination with NY-ESO-1 and ISCOM provided therapeutic efficacy to cancer patients. The data described herein elaborate on this work, as will now be explained.

While it is well known that tumor specific CD8⁺ T cells play an important role in tumor immunosurveillance, CD8⁺ T cell activation generally requires help from CD4⁺ cells. See, e.g., Cella, et al., J. Exp. Med., 184:747-752 91996); Wang, et al., Trends Immunol., 22:269-276 (2001). This is generally true for tumor immunity, and autoimmunity, and may be an important pathway for tumor-specific immunity in the case of, e.g., “cross-priming.” See, Yu, et al., J. Exp. Med., 197:989-995 (2003). Cognate help received from CD4⁺ T cells, by APCs, may be essential in this mechanism. See, Bennett, et al., J. Exp. Med., 186:65-70 (1997).

The mechanism of “T help” have been the focus of more extensive investigation recently. T helper cells may help CD8⁺ T cell priming through upregulation of their CD40 ligand expression, which in turn interacts with CD40 molecules expressed on professional APC, most likely dendritic cells (DCs), to “license” (8) them for priming naive CD8⁺ T cells, (Bennett, et al., Nature, 393:478-480 (1998); Schoenberger, et al., Nature, 393:480-483 (1998); Ridge, et al., Nature, 393:474-478 (1998). Further, CD4⁺ T cells may also help CD8⁺ T cells through providing general growth factors (such as IL-2, (see Fearon, et al., Cell, 60:397-403 (1990)), to promote CD8⁺ T cell activation and proliferation. Additionally, CD4⁺ T cells also play very important roles post licensing DCs, including direct effector functions such as secreting IFNγ (Christensen, et al., Proc. Natl. Acad. Sci. USA, 96:5135-5140 91999); Marzo, et al., J. Immunol., 165:6047-6055 (2000) and cytotoxicity. More recently, CD4⁺ T cells were shown to be necessary in a memory response for CD8⁺ T cells to become fully activated (Gao, et al., Cancer Res., 62:6438-6441 92002)), to sustain their functionality (Cardin, et al., J. Exp. Med., 184:863-871 (1996)) and to expand efficiently (Janssen, et al., Nature, 421:852-856 (2003)). Through gene knockout and in vivo antibody-mediated depletion experiments, the necessity of the CD4⁺ T cells was also demonstrated in general immune responses to tumor (Marzo, et al., supra) and to some viral antigens (Matloubian, et al., J. Virol., 68:8056-8063 (1994)). In murine immunological experiments a T helper determinant was often incorporated in the immunization (Vitiello, et al., J. Immunol., 157:5555-5562 (1996)) to enhance CD8⁺ T cell induction. This is now recommended as a general consideration for better anti-viral and anti-tumor therapeutics (Zajac, et al., Curr. Opin. Immunol., 10:444-449 (1998); Yu, et al., J. Clin. Invest., 110:289-294 (2002)). The majority of trials; however, have so far failed to reveal any general practical strategies and the clinical outcomes are more disappointing than encouraging. One of the potential design flaws might be that not enough emphasis has been given to the CD8⁺ and CD4⁺ T cell interaction. More likely a successful vaccine will be either full-length tumor antigen or antigens incorporating robust CD4⁺ help (Zeng, et al., Cancer Res., 62:3630-3635 (2002)). The latter presents a greater challenge to design a universal vaccine which takes the polymorphic requirements of various MHC Class I and II molecules into account yet at the same time provide the “danger” signal to trigger the immune system. See, Matzinger, et al., Annu. Rev. Immunol., 12:991-1045 (1994).

As noted, supra, it has been frequently observed that patients who develop anti-NY-ESO-1 antibodies normally have detectable CD8⁺ T cell responses (Jager, et al., Proc. Natl. Acad. Sci. USA, 97:4760-4764 (2000); Zeng, et al., Proc. Natl. Acad. Sci. USA, 98:3964-3969 (2001)). More recently the observation has been extended to CD4⁺ T cells (Gnjatic, et al., Proc. Natl. Acad. Sci. USA, 100:8862-8867 (2003)). The invention describes a novel vaccine formulation comprising NY-ESO-1 antigen and a saponin, and identifies and characterizes novel CD4⁺ T cell determinants from a NY-ESO-1 vaccinated patient.

Since the identification of the first human tumor CD8⁺ T cell determinant in the early 1990s there have been more than 150 CD8⁺ T cell determinants and a few dozen CD4⁺ determinants characterized (for reviews, see Renkuist, et al., Cancer Immunol. Immunother, 50:3-15 (2001); Davis, et al., J. Leukoc. Biol., 73:3-29 (2003)). Among the CD8⁺ T cell determinants the majority are presented by tumor cells or tumor derived cell lines. Many of the defined minimum CD8⁺ T cell determinants have been used as antigens for peptide-based vaccine trials worldwide (Yu, et al., supra; Davis, et al., supra; Jager, et al., Curr. Opin. Immunol., 14:178-182 (2002)).

Hence features of the invention which will be seen herein include immunogenic compositions of NY-ESO-1 and a saponin, which is well tolerated, highly immunogenic, and which induces humoral (Ab) and T cell (both CD4⁺ and CD8⁺) immune responses in patients who received it. The patients who received the immunogenic composition showed a clinical outcome superior to those patients receiving placebo, or NY-ESO-1 protein alone.

### BRIEF DESCRIPTION OF THE FIGURES

FIG. 1 shows data obtained from patient studies, intended to determine DTH reactions and their extent.

FIG. 2 shows a summary of results obtained from experiments designed to determine antibody responses.

FIG. 3 compares ELISA results using recombinant NY-ESO-1 from bacterial and mammalian cells.

FIG. 4 presents the results of experiments using NY-ESO-1 based 13 mer peptides, as discussed in Example 5.

FIG. 5 presents the results of additional 13 mer peptides. See Example 5.

FIG. 6 shows results with 13 and 18 mer peptides, to determine CD4⁺ and CD8⁺ epitopes.

FIG. 7 shows the results of the comparison of the time to relapse, in patients receiving the immunogenic composition of the invention, and those who did not.

### EXAMPLE 1

This example describes the in vivo study used to test the formulation described supra. In brief, it was a double blind, placebo controlled, phase I dose escalation clinical trial.

Eligible patients were defined as those who had previously exhibited a cancer that expressed NY-ESO-1, as determined either by immunohistochemistry, or RT-PCR. Patients had minimal residual disease (i.e., no detectable disease, or small volume, locoregional disease only), and a relapse risk of at least 25% within 5 years). Further, patients had to have no other effective therapy available, or appropriate, an expected survival time of at least 3 months, and had to have received no immunodeficiency or immunosuppressive therapy.

Five dose levels were used: dose level A was 10 μg of NY-ESO-1 protein in 12 μg ISCOM (3 patients); dose level B was 36 μg of the protein in 36 μg ISCOM (3 patients); dose level C was 100 μg of protein in 120 μg ISCOM (16 patients, divided equally between HLA-A2 positive and negative patients), and dose level D was 100 μg NY-ESO-1 without ISCOM (16 patients, equally divided between HLA-A2 positive and negative patients). Randomization was in effect for dose levels C and D, such that four additional patients in each group, equally divided between HLA-A2 positive and negative patients, received sterile saline as placebo.

The dosing regime consisted of three intramuscular injections, at 4 week (28 day) intervals, as well as two, 1 μg intradermal injections, for DTH testing.

### EXAMPLE 2

Patients were examined for DTH reactions, at the baseline of the study, and at the 84^{th} day. Two days after the injection of the 1 μg of NY-ESO-1 protein (i.e., at days 2 and 86), induration and erythema were measured. These measurements were taken before and after the vaccinations. Pre-existing reactivity was defined as a baseline induration of at least 6 mm. A positive response to vaccination was defined as one where the second reading was at least 6 mm, and at least double the baseline.

Patients who received vaccines commonly developed DTH responses, especially when receiving dose level C. Some significant DTH responses were observed. These responses were characterized by erythema and induration. Biopsies of the reactions showed dermal, lymphoid infiltrates, consisting primarily of CD4⁺ T cells, and a lesser population of CD8⁺ T cells. The specificity of the CD8⁺ and CD4⁺ T cells infiltrate was assessed in one of these DTH positive patients. Isolated infiltrating lymphocytes were tested for recognition of a panel of overlapping 18-mer peptides covering the entire NY-ESO-1 amino acid sequence. Recognition of the NY-ESO-1 peptides was confirmed, using an intracellular IFNγ staining assay, as taught by Jung T., et al., J. Immunol. Methods, 159:197-207 (1993), incorporated by reference. The NY-ESO-1/ISCOM immunogenic composition exhibited an enhanced DTH response as compared to the NY-ESO-1 protein, with 11/16 of the patients receiving dose level C of the immunogenic composition responding, as compared to 1/16 of those who received dose level D, i.e., NY-ESO-1 protein with no ISCOM adjuvant.

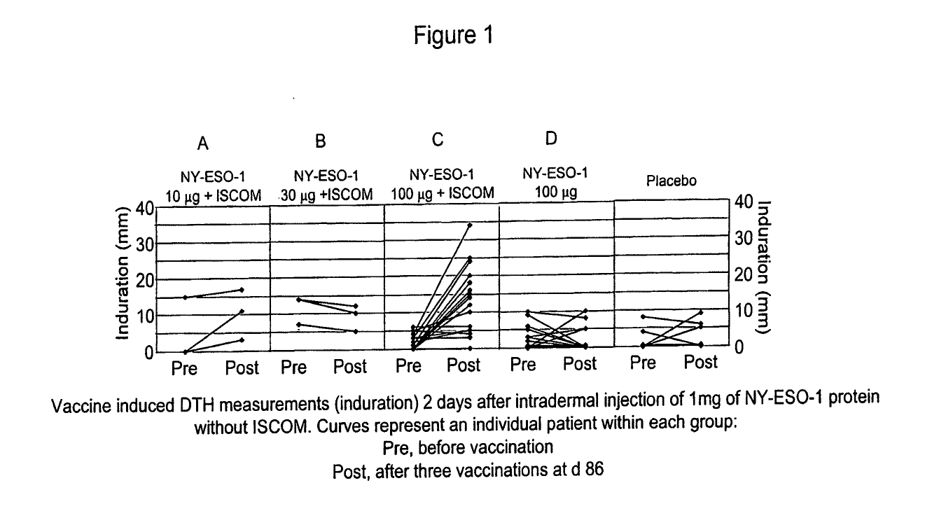

In dose level A, one patient had a pre-existing DTH response, which, as expected, did not change following vaccination. See Jager, et al., Proc. Natl. Acad. Sci. USA, 97:12198(2000), referred to supra. One additional patient developed a positive response. All patients in dose level B had pre-existing response, and these did not change following administration, again, as expected. All of these results are set forth in FIG. 1.

Of the total of 46 patients, nine had pre-existing DTH responses. With respect to the 8 members of the placebo groups, one had pre-existing reactivity, while a second one developed a 9 mm induration, and 30 mm erythema after the second DTH injection. Both of these patients were HLA-A2 negative, and did not develop antibody responses, as discussed supra.

### EXAMPLE 3

Subjects were also tested to determine if they had developed antibody responses to NY-ESO-1. The assays were carried out in a standard ELISA, as taught by Stockert, et al., J. Exp. Med., 187:1349 (1998). In brief, the capture antigen was the same, purified, NY-ESO-1 protein used in the manufacture of the vaccine. The detection antibody was horseradish peroxidase labeled, affinity purified, goat anti-human IgG. The assay was carried out at 5 points in time, i.e., before vaccination and then at days 14, 42, 70 and 86.

Patients who had a pretreatment titer greater than 5000 were deemed to have a pre-existing response, while patients with a pretreatment titer below 5000, who developed a titer above 5000 at any point following vaccination humoral, were deemed to have a positive antibody response to NY-ESO-1.

In all, three of the patients had a pre-existing antibody titer above 5000, which did not change significantly during the vaccination protocol. All patients who received the immunogenic composition of NY-ESO-1 protein and ISCOM adjuvant (i.e., dose B or C), developed a positive response, while 4/16 of the patients who received NY-ESO-1 protein without adjuvant (dose D) developed such a response. These 4 responses were generally of a much lower titer than those of the patients who received the immunogenic composition of NY-ESO-1 protein and adjuvant, indicating the importance of the immunogenic composition in the response. Western blot assays provided additional confirmation that patient sera could recognize NY-ESO-1.

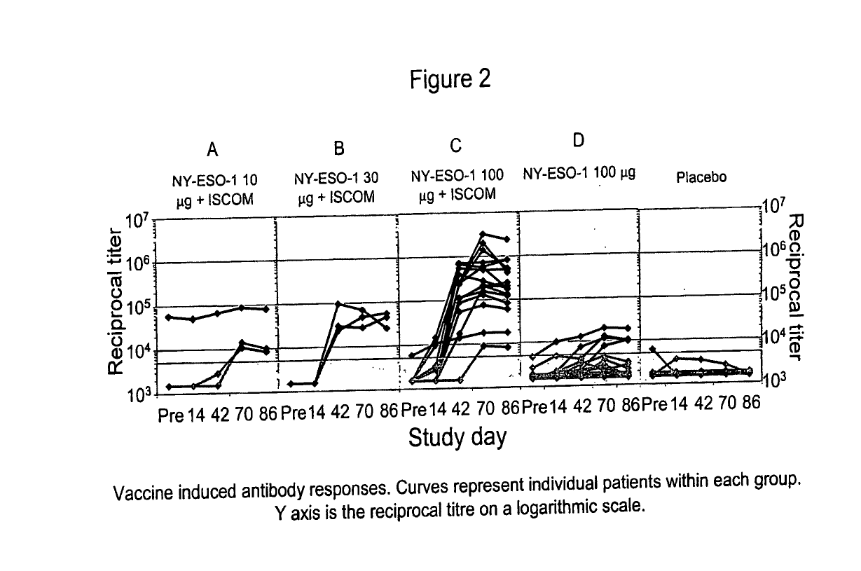

No placebo patients developed an antibody response during the study. FIG. 2 summarizes these results.

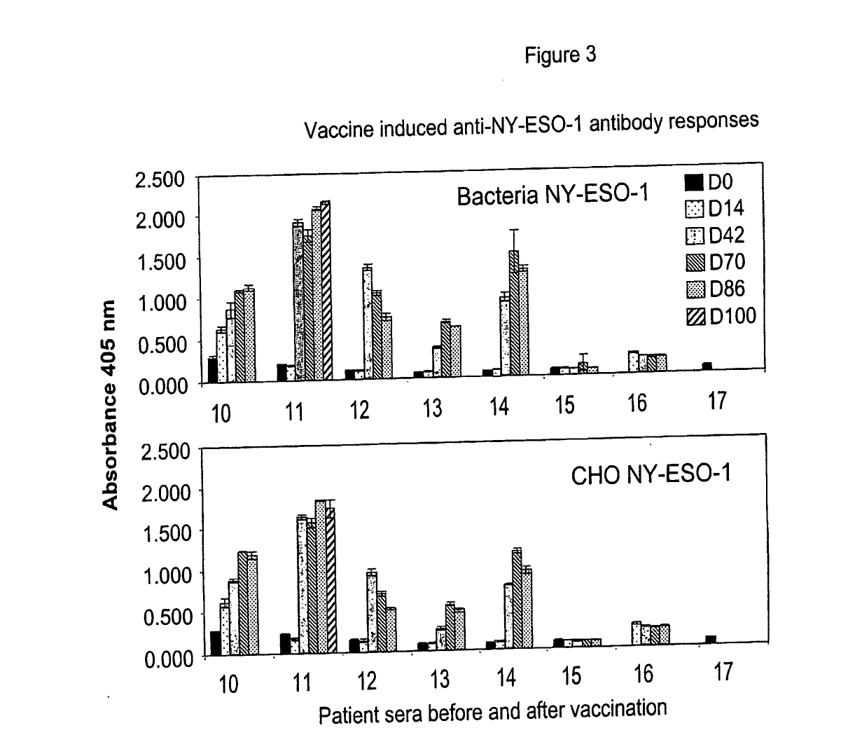

In an additional analysis, a series of standard ELISA experiments were carried out on samples from a selection of vaccinated patients, where a recombinant, NY-ESO-1 protein was used which had been produced in a standard mammalian cell line, i.e., a CHO cell line, and compared to ELISAs carried out using NY-ESO-1 produced in E. coli. Consistent results were obtained using both recombinant NY-ESO-1 proteins, as will be seen in FIG. 3.

### EXAMPLE 4

These experiments were designed to measure T cell responses of the patients in the study.

Initial T cell assays were restricted to those patients who were HLA-A2⁺. The method used, taught by Jung, et al., J. Immunol. Meth., 159:197-207 (1993), incorporated by reference, is a modified, flow cytometric or intracellular cytokine staining (“ICS”) assay, designed to measure T-cell intracellular, interferon gamma expression, together with HLA-peptide tetramer binding.

In brief, 5×10⁶ peripheral blood mononuclear cells were taken from HLA-A2 positive patients, using standard methods. These were then pulsed with a peptide consisting of amino acids 157-163 of the NY-ESO-1 protein, as seen in, e.g., U.S. Pat. No. 6,525,177, SEQ ID NO: 8, incorporated by reference herein. Peptide pulsing was carried out, using 0.1 μM of peptide in the presence of 250 μM 2-carboxyethyl phosphine hydrochloride (“TCEP”), for 30 minutes, at room temperature. Cells were then washed, and cultured in a 24 well plate in 2m1 of RPMI, containing 10% fetal calf serum, and 10 IU/ml of IL-2.

Cells were harvested after 7 days of culture, and assayed for intracellular gamma interferon expression, as compared to T2 cells which had been pulsed with the same peptide.

For HLA-A2 patients, binding of tetrameric HLA-A2/NY-ESO-1 peptide 157-163 complexes, prepared in accordance with Jager, et al., Proc. Natl. Acad. Sci. USA, 97:4760-4765 (2000) were used to detect CM⁺ T cells specific for the NY-ESO-1 157-163 peptide.

Five patients showed a positive CD8⁺ T cell response. Of these, one patient received dose “A”, three dose “C”, and one dose “D”, i.e., ISCOM and 10 μg protein, an immunogenic composition of ISCOM and 100 μg NY-ESO-1 protein, and 100 μg NY-ESO-1 protein alone. The patient who received dose A displayed a pre-existing antibody response, as did one of the patients receiving dose C. A second patient receiving dose C had a pre-existing DTH response. The detection of a NY-ESO-1 specific T cell response to the NY-ESO-1 157-163 peptide was consistent with both the ICS and tetramer staining assays

### EXAMPLE 5

These experiments were designed as follow up to the experiments in Example 4.which measured NY-ESO-1 T-cell responses to a single CD8⁺ T-cell peptide antigen. Specifically, they were designed to determine if broader CD8⁺ and CD4⁺ T cell responses to both MHC-Class I and Class II epitopes from NY-ESO-1 had been induced by the NY-ESO-1/ISCOM immunogenic composition.

The methodology described in Example 4, supra, was used, except TCEP was omitted, as were T2 cells. Autologous peripheral blood mononuclear cells (“PBMCs”) were used in place of T2 cells. A series of overlapping NY-ESO-1 peptides were synthesized, using standard methods. These fell into two groups: 18 mers, overlapping by 6 amino acids, which were used as stimulating peptide antigens, while 13 mers, overlapping by 2 amino acids, were used to pulse target cells in specificity assays. Each 18 mer peptide was pulsed onto autologous PMBCs, and stimulated in vitro CD4⁺ and CD8⁺ T-cells were measured for specificity using the 13 mer peptides as described supra.

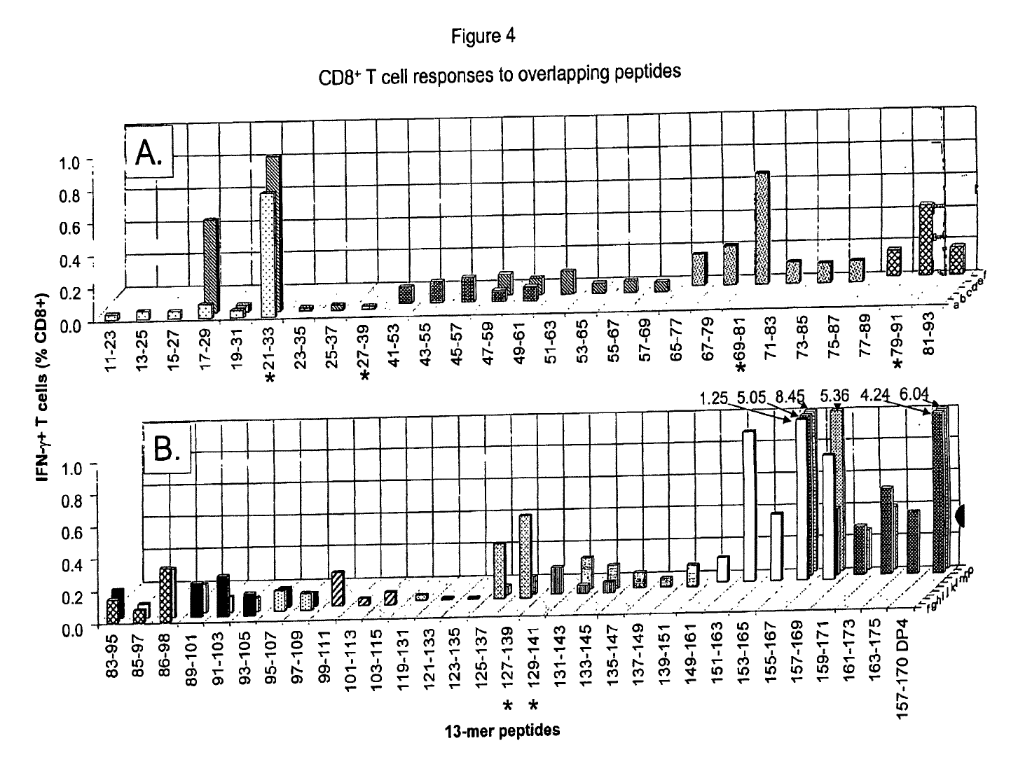

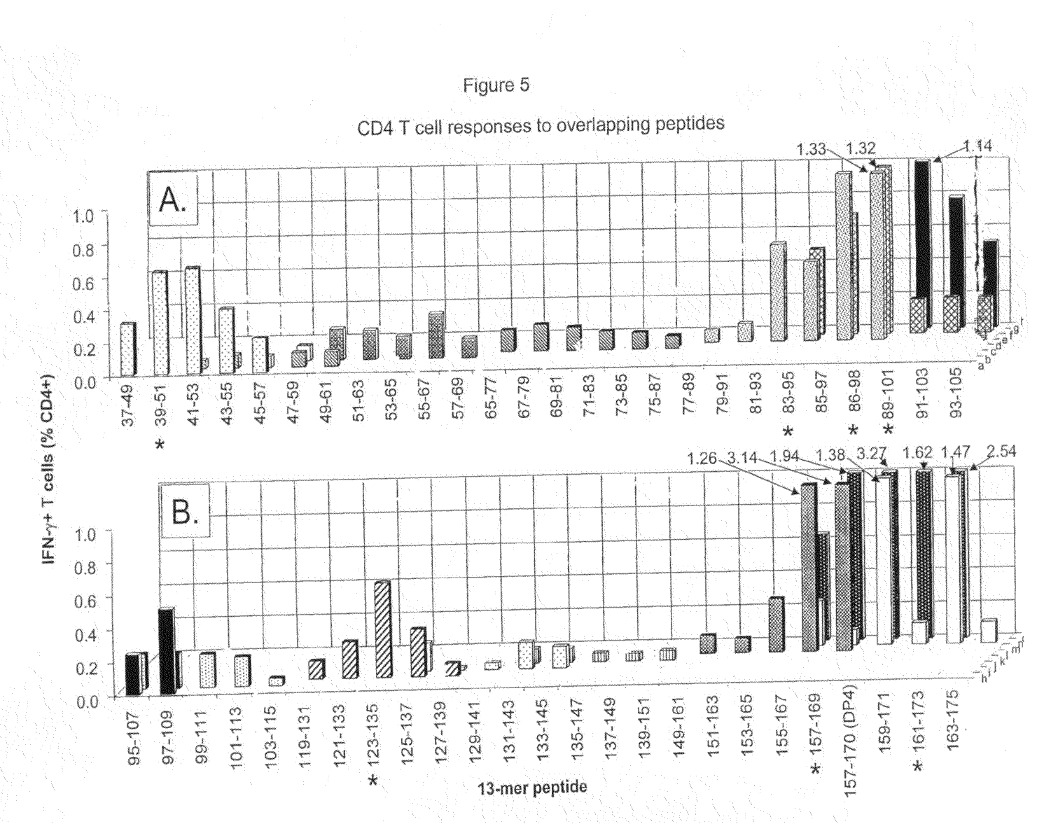

Results from one representative patient are shown in FIGS. 4 and 5, which present T cell recognition of the NY-ESO-1 for 13 mers. FIG. 4 shows the results for CD8⁺ T cells and FIG. 5, for CD4⁺ cells. The amino acid residue numbers for the NY-ESO-1 13 mers are presented by the X axis. With respect to 18 mers, used for stimulation, these are presented on the Y axis with letters corresponding to amino acid numbers for the complete sequence of NY-ESO-1 (SEQ ID NO: 1), as follows:

| Letter (FIGS. 1 and 2) | Amino acids |
|---|---|
| a | 13-30 |
| b | 19-36 |
| c | 43-60 |
| d | 49-66 |
| e | 67-84 |
| f | 79-86 |
| g | 85-102 |
| h | 91-108 |
| i | 97-114 |
| j | 121-138 |
| k | 127-144 |
| l | 133-150 |
| m | 151-168 |
| n | 157-174 |
| o | 157-170 |

In FIG. 5, the letters correspond to the same amino acid residues, except “aa” is new, and corresponds to NY-ESO-1 amino acids 37-54. Novel epitopes are marked by an asterisk.

A more complete listing of the peptides follows:

CD8⁺ T cell epitopes:

| Amino acids | Peptides | Comment |
|---|---|---|
| 21-33 | PGIPDGPGGNAGG | Novel |
| 17-29 | GPGGPGIPDGPGG | Novel |
| 69-81 | ASGLNGCCRCGAR | Novel |
| 79-91 | GARGPESRLLEFY | Novel |
| 127-139 | TVSGNILTIRLTA | Novel |
| 129-141 | SGNILTIRLTAAD | Novel |
| 151-163 | SCLQQLSLLMWIT | Contains 155-163 |
| | | HLA-A2 epitope |
| 157-165 | SLLMWITQC | HLA-A2 epitope |
| 157-169 | SLLMWITQCFLPV | Contains known |
| | | HLA-A2 epitopes |
| | | (157-165; 157-167; |
| | | 159-162) |

CD4⁺ T cell eptiopes

| Amino acids | Peptide | Comment |
|---|---|---|
| 39-51 | ATGGRGPRGAGAA | Novel |
| 85-97 | SRLLEFYLAMPFA | Novel |
| 89-101 | EFYLAMPFATPME | Novel |
| 123-135 | LKEFTVSGNILTI | Novel |
| 157-169 | SLLMWITQCFLPV | Novel^{a} |
| 157-170 | SLLMWITQCFLPVL | HLA-DP4 epitope^{a} |
| 161-173 | WITQCFLPVFLAQ | Novel |

^{a}One amino acid short of HL4-DP4 157-170 epitope. Zang, et al., Proc. Natl. Acad. Sci. USA, 98: 3964-3969 (2001).

The results, from a single representative patient, show clear evidence that there is a broad range of circulating NY-ESO-1 specific CD4⁺ and CD8⁺ T cells in the peripheral blood of vaccinated patients receiving the immunogenic composition, specific for multiple, NY-ESO-1 epitopes, both known and unknown until now.

In additional, follow-up experiments, samples taken from six patients who had received dose C (i.e., the immunogenic composition of NY-ESO-1 protein plus ISCOM adjuvant), were analyzed in the same way, i.e., the T cell samples were mapped with 18 mer/13 mer NY-ESO-1 peptides, as described supra.

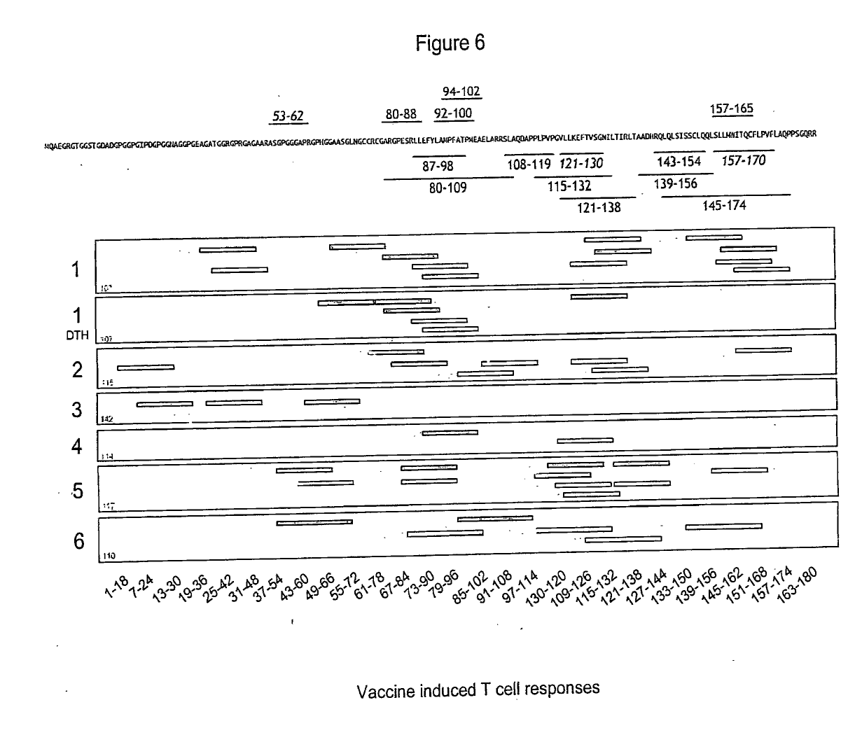

FIG. 6 shows the results of this T-cell recognition analysis of the additional patients. CD4⁺ T-cell epitopes are in light boxes, while CD8⁺ T-cell epitopes are in the dark boxes. The amino acid sequence at the top of the figure in the amino acid sequence for NY-ESO-1 as used throughout this application. Previously defined NY-ESO-1 epitopes may be seen, supra, as well as in Gnjatic, et al., Proc. Natl. Acad. Sci. USA, 100:8862-8887 (2003), incorporated by reference.

Many of these detected T cell responses were induced by the vaccine, as the patients had no pre-existing immune response to NY-ESO-1. Spontaneous or naturally induced responses to some of these epitopes have been described previously in cancer patients. See Gnjatic, et al., supra, Jager, et al., Proc. Natl. Acad. Sci. USA, 97:4760-4765 (2000); and Zeng, et al., Proc. Natl. Acad. Sci. USA, 98:3964 (2001), indicating that the NY-ESO-1/ISCOM immunogenic composition induces T cell response to epitopes that are naturally processed. The results also indicated that the vaccine induced T cell responses to novel peptide epitopes for both CM⁺ and CD4⁺ T cells.

### EXAMPLE 6

Forty two melanoma patients completed the vaccination schedule, 3 months of treatment. There were 13 total patients who received dose C of the immunogenic composition (NY-ESO-1 and ISCOM). Of these, only one has relapsed, with a median follow up of 709 days, over a range of 283-1000 days. As discussed, supra., these vaccinated patients also have immunogenic composition induced Ab and T cell immunological responses to NY-ESO-1. In contrast, 6/16 patients who received dose D (NY-ESO-1 protein alone) have in fact relapsed, as have 5/7 patients who received the placebo. These patients had less of an immune response to NY-ESO-1 overall. After a median follow up of 748 days, 5/7 patients receiving the placebo and 6/16 patients who received dose D (NY-ESO-1 protein alone) had relapsed. A total of nineteen patients had received the immunogenic composition of NY-ESO-1/ISCOM adjuvant (dose A, B & C), and only 2/19 had relapsed.

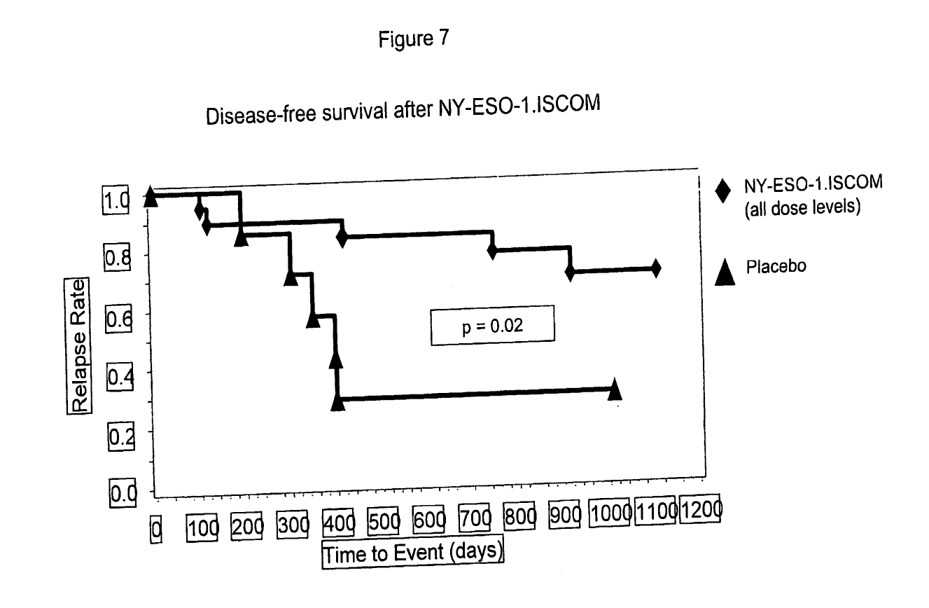

Comparison of all patients receiving the NY-ESO-1/ISCOM immunogenic composition, versus placebo receiving patients, showed a significant difference in time to relapse (p=0.02), as is shown in FIG. 7. After adjusting for co-variants, there were no significant differences between the populations, with respect to pathological stage at study entry, primary lesion thickness, age, sex, time since diagnosis, estimated risk of relapse at study entry, number of recurrences before entry, and time since last resection.

One year after this initial analysis, there have been 5/19 relapses in the group of patients receiving the NY-ESO-1/ISCOM immunogenic composition. There has been one additional relapse in the group receiving NY-ESO-1 protein alone (dose D), or 7/16. The placebo group remains as it was a year previously, at 5/7 relapses.

### EXAMPLE 7

In this, and the experiments which follow, additional CD4⁺ T cell determinants, as well as their use, are described. In a first step, autologous monocyte derived dendritic cells (MoDCs) were obtained from a subject to carry out in vitro stimulation assays, as explained infra. MoDCs were used because of their ability to take up exogenous antigens, and to present them to both CD4⁺ and CD8⁺ T cells.

To generate the MoDCs, the method of Luft, et. al., J. Immunol., 167:2529-2537 (2001), incorporated by reference, was used. In brief, CD14⁺ cells were isolated from the sample, using anti-CD4-conjugated, MACs beads, and were then cultured in medium containing GM-CSF (20 ng/ml), and IL-4 (500 U/ml), for 7-8 days. This resulted in immature MoDCs, which expressed limited amounts of CD80 and CD83.

The MoDCs were then loaded with 10-20 μg/ml of the vaccine described supra, at 37° C., for 2 hours. The cells were then contacted with TNFα (20 ng/ml), INFa (1000 U/ml), and prostaglandin E2 (TNP) (1 μM), and incubated at 37° C., for 2 more hours. The treatment with TNP pushed the MoDCs to maturation, i.e., they expressed high levels of CD80 and CD83. In addition, they expressed higher levels of CD86 and HLA-DR.

### EXAMPLE 8

The mature MoDCs, described in the prior example, were then used to generate CD8⁺ T cells specific for the NY-ESO-1 peptide consisting of amino acids 157-165.

To do this, autologous, CD14-PBMCs, i.e., cells taken from the same patient as the CD14⁺ cells, were combined with the MoDCs that had been loaded with the vaccine (DC:PMBC ratio: 1:10) in the presence of IL-2 (10 U/ml). The culture was replenished with fresh medium every 2-3 days, and split as required by cell density. Cells were collected after 10-13 days, and were screened against 18 mer and 13 NY-ESO-1 peptides. The production of INFγ was measured, as a determination of CD8⁺ T cells.

The results indicated that these cells, i.e., the MoDCs were in fact able to stimulate T cell proliferation.

### EXAMPLE 9

Next, CD4⁺ and CD8⁺ T cell responses to NY-ESO-1 were examined. Specifically, the immature MoDCs referred to supra were loaded with the vaccine as described supra. The MoDCs were then pushed to maturity with TNP, also as described supra, and co-cultured with thawed CD14-PBMCs from the same patient for about 10 to 15 days, also as described supra, to generate T cells. The resulting T cells were screened with a set of 18mer NY-ESO-1 peptides covering the whole NY-ESO-1 sequence. The NY-ESO-1 peptides overlapped each other by 12 amino acids.

This screening was accomplished by using autologous Epstein Barr virus transformed B lymphocyte cell lines (BLCLs) as the antigen presenting cells (“APCs”). These were established from the patient from whom the MoDCs and PBMCs were obtained. The intracellular cytokine staining (ICS) method of Jung, et. al., J. Immunol. Methods, 159:197-207 (1993), incorporated by reference, was used. In short, BLCLs were pulsed with 1 μM concentration of peptide, in the presence of 10% fetal calf serum (FCS) at 37° C. for 2 hours to allow for serum-mediated processing, as described in Sherman, et. al., J. Exp. Med., 175:1221-1226 (1992), incorporated by reference, and potential antigen uptake. Bulk cultured T cells and Brefeldin A (BFA, 10 μg/mL) were then added for an additional 4 hours before the cells were harvested and stained with anti-CD4-PE or anti-CD8-Cychrome in PBS. The cells were then washed and fixed with 1% paraformaldehyde in PBS. The cells were further stained with anti INFγ in the presence of 0.2% saponin. Samples of 100,000 cells were analyzed.

The culture contained only approximately 6% of CD8⁺ T cells. These cells were clearly identifiable, antigen-specific CD8⁺ T cells, but they were further diluted as a result of a larger amount of CD4⁺ T cell expansion.

### EXAMPLE 10

Due to the much greater CD4⁺ T cell expansion resulting from the experiments described in Example 9, efforts focused on the CD4⁺ T cells, in the experiments which follow.

Antigen specific CD4⁺ T cells that were stimulated as described supra were assessed for their specificities against a set of 18mer NY-ESO-1 peptides covering the whole NY-ESO-1 sequence described supra using autologous BLCLs as APCs, also as described supra. These peptides were incubated with BLCLs in the presence of FCS, at room temperature, for 60 minutes. CD4⁺ T cells and BFA were then added for an additional 4 hours before harvesting and standard ICS as described supra.

The results showed that the strongest CD4⁺ responses came from BLCLs pulsed with NY-ESO-1 peptides consisting of amino acids 85-102 and 157-174.

Following the results obtained using the 18mers set, the same CD4⁺ T cell cultures were screened, using the same method described supra, against a 13mer NY-ESO-1 peptide set covering the whole NY-ESO-1 sequence, where the peptides tested had 11 amino acid overlaps. This additional screening was performed to acquire an independent and more accurate assessment for the core sequences of the presented peptides.

The results indicated that the strongest responses were to peptides consisting of amino acids 85-97, and 157-169 of NY-ESO-1.

### EXAMPLE 11

After the detection of the CD4⁺ T cell determinants which elicited the strongest responses in Example 10, efforts focused on HLA restriction, i.e., which HLA molecule presents these CD4⁺ T cell determinants.

HLA restriction was determined by using the autologous BLCLs described supra, and pulsing them with 1 μM peptides consisting of amino acids 85-102 or 157-174 of NY-ESO-1 at 37° C. for 1 hour. The cells were then washed and 20 μL anti HLA-Class II antibody supemate was added for an additional hour. The CD4⁺ T cells used in Examples 9 and 10 supra and BFA were then added for 4 hours before harvesting by standard methods. The production of INFγ from activated T cells was measured in a standard ICS assay described supra.

In these blocking assays, anti-DR antibody efficiently blocked the T cell response to the peptide consisting of amino acids 85-102 of NY-ESO-1, whereas anti-DP antibody blocked the T cell response to the peptide consisting of amino acids 157-174 of NY-ESO-1.

Peptides consisting of amino acids 157-169 of NY-ESO-1 represents a previously identified CD4⁺ cell determinant restricted by HLA-DP4. Since the patient is DP4 positive, the strong response to peptides consisting of amino acids 157-169 of NY-ESO-1 confirms the previously identified CD4⁺ cell determinant; however, the fact that T cells recognized the peptide consisting of amino acids 85-102 of NY-ESO-1 is a novel finding, i.e., that a peptide consisting of amino acids 85-102 was recognized by DR-restricted, CD4⁺ cells.

### EXAMPLE 12

To further identify the restricting DR molecules, Epstein Barr Virus transformed B lymphocyte cell lines (BLCLs) expressing homozygous HLA-DR alleles (DR1⁺9080; DR2⁺ T242) identical to the patient were obtained. Also, BLCLs expressing homozygous HLA-DR alleles (DR6⁺/DR7⁺: T282) but void of HLA-DR1⁺ and HLA-DR2⁺ alleles were used, as well as LCLs (?) expressing autologous heterozygous HLA-DR alleles (DR1⁺/DR2⁺). Bulk T cells originally stimulated by MoDCs described in Example 1, were further stimulated with peptides consisting of amino acids 85-102 of NY-ESO-1 and tested on various APCs pulsed with this peptide, i.e., APCs homozygous for DR2, homozygous for DR1⁺ APC, autologous heterozygous for DR1⁺/DR2⁺ APCs, and APCs with HLA-DR alleles but void of DR1⁺ or DR2⁺.

The results showed that the greatest response from T cells specific for peptides consisting of amino acids 85-102 of NY-ESO-1 came from the T cells' response to the autologous heterozygous LCLs (DR1⁺/DR2⁺). The results also indicated that the majority of these antigen specific T cells were DR-2 restricted. Further, the homozygous DR1⁺ cell line stimulated about 10% of the total antigen-specific T cells, which could potentially explain the greater responses to autologous heterozygous versus homozygous DR2⁺ APC. Finally, as expected, the APCs which were void of either DR1⁺ or DR2⁺ alleles elicited the smallest response.

To address the possibility of multiple CD4⁺ T cell determinants within the same peptide, i.e., peptides consisting of amino acids 85-102 of NY-ESO-1, T cell sub-lines were derived from the bulk T cell lines discussed in the prior example, and then were tested against the homozygous DR2⁺, and DR1⁺ lines, as well as the heterozygous DR1⁺/DR2⁺ line.

The results indicated that one of the sub-lines showed exclusive DR1-restriction for all of the T cells specific for peptides consisting of amino acids 85-102 of NY-ESO-1. Therefore, there are at least two CD4⁺ T cell determinants within the peptides consisting of amino acids 85-102 of NY-ESO-1, i.e., one presented by DR2⁺ and one by DR1⁺.

### EXAMPLE 13

To further identify the minimum CD4⁺ T cell determinant sequence, 13mer peptides found within amino acids 85-102 of NY-ESO-1 were titrated and core sequences consisting of amino acids 85-97 and 89-101 of NY-ESO-1 were located using either DR1-restricted or DR2-restricted T cell sublines.

Then, extended and truncated peptides based on the 13mer core sequences were synthesized and were used to pulse homozygous, autologous PMBC in the absence of FCS for 1 hour to avoid serum-mediated processing. Dose dependent titration was used. Excess peptides were washed before the addition of antigen specific T cells. Standard ICS assays were performed, as described supra.

The results indicated that the minimum yet most potent sequence for the DR-1-restricted T cell determinant was the peptide consisting of amino acids 89-100 of NY-ESO-1. The minimum sequence for the DR2-restricted T cell determinant was the peptide consisting of amino acids 86-99 of NY-ESO-1. The T cells specific for this latter determinant did not recognize the DR1-restricted minimum peptide consisting of amino acids 89-100 of NY-ESO-1. When variants were prepared which included amino acid 88, and were tested in the same manner described supra, a strong response was observed, indicating that amino acid 88 was essential for binding to DR2⁺ cells.

### EXAMPLE 14

After acquiring the minimum sequences for the CD4⁺ T cell determinants described in Example 7, efforts focused on determining how important these determinants were in the CD4⁺ T cell immununodominance hierarchy.

To address this question, multiple PMBCs taken from the patient, collected at various times, post vaccination, were thawed and divided into two parts. These cells were stimulated with either the reported minimum DP4-restricted peptide, i.e., a peptide consisting of amino acids 157-169 of NY-ESO-1, or the DR2-restricted peptide, consisting of amino acids 86-99 of NY-ESO-1. The antigen specific T cell percentages were analyzed on day (11 or 14?) in a standard ICS assay, as described supra.

The results show that the DR2-restricted CD4⁺ T cell response was detected earlier and was greater in magnitude than the DP4-restricted CD4⁺ T cell response. Further, compared with an earlier analysis for specific CD8⁺ T cell responses to peptides consisting of amino acids 157-165 of NY-ESO-1, the DR2-restricted CD4⁺ T cell response was detectable at the same time as the earliest detectable aforementioned CD 8⁺ T cell response.

Thus, the newly identified DR2-restricted CD4⁺ T cell determinant, i.e., amino acids 86-99, was immunodominant.

### EXAMPLE 15

Next, polyclonal T cell receptor (TCR) usage of the novel CD4⁺ T cells specific for peptides consisting of amino acids 86-99 of NY-ESO-1 was measured. PMBCs collected from the patient on day 86 post vaccination were stimulated with either the DP4-restricted peptide, the peptide consisting of amino acids 157-170 of NY-ESO-1, or DR2-restricted peptide, the peptide consisting of amino acids 86-99 of NY-ESO-1. The T cells were then activated and stained in an ICS assay plus single Vβ antibodies. Vβ positive and antigen specific T cells were displayed as % of total antigen-specific T cells.

The results show that the novel CD4⁺ T cells specific for peptides consisting of amino acids 86-99 of NY-ESO-1 had a broader TCR usage than the previously identified DP4-restricted CD4⁺ T cells.

### EXAMPLE 16

It was hypothesized that the newly identified T cell determinants described supra represented naturally presented determinants. The CD4⁺ T cell line specific for peptides consisting of amino acids 85-102 of NY-ESO-1 was used to read out antigen presentation of autologous MoDCs, described supra, pulsed with the vaccine described supra (10 μg/ml).

The results show that both the DR1 and DR2-restricted CD4⁺ T cell determinants were presented by the autologous MoDCs loaded with the vaccine. In other words, after being pulsed with the full length protein plus an adjuvant, these DCs processed the full length protein and naturally presented the newly discovered NY-ESO-1 determinants.

Additionally, the CD4⁺ T cell line specific for peptides consisting of amino acids 85-102 of NY-ESO-1 was used to read out antigen presentation of a DR1⁺ melanoma cell line NW-Mel-38, described in Jager, et. al., J. Exp Med., 191:625-630 (2000), incorporated by reference. This CD4⁺ T cell line was also used to read out antigen presentation of a DR2⁺ allogenic melanoma cell line LAR 1. Both of these melanoma cell lines were cultured with “RP-10” consisting of RPMI-1640 supplemented with 10% FCS, L-glutamine (2 mM), 2-ME (5×10⁻⁵ M) and antibiotics (penicillin 100 U/ml, streptomycin 100 ug/mL). Further, both DR1⁺ and DR2⁺ cell lines were cultured with and without 100 ng/ml recombinant human INFγ for 48 hours.

The results show that the CD4⁺ T cell line specific for peptides consisting of amino acids 85-102 of NY-ESO-1 recognized naturally presented NY-ESO-1 determinants presented by both melanoma cell lines after INFγ treatment in vitro. The CD4⁺ T cell line was not activated by either the above cell lines without INFγ induction, or as predicted, by tumor cells that did not express the appropriate DR allele.

### EXAMPLE 17

For several patients in the clinical study, matched pre-vaccination tumor samples and relapsed tumor samples following immunogenic composition vaccination, were available. Expression of the NY-ESO-1 antigen, HLA-class I heavy chain and β₂-microglobulin (β₂M) is critical for presentation of NY-ESO-1 CD8⁺ T cell peptide epitopes by the tumor cells and recognition by NY-ESO-1 specific T cells in the patients who received the composition. Expression of these three molecules was analyzed by standard immunohistochemical analysis using appropriate monoclonal antibodies, in the matched tumor samples. Six patients were investigated. Four patients received the NY-ESO-1.ISCOM immunogenic composition (1 dose A, 3 dose C) and for each of them the relapsing tumor showed a significant decrease or loss of expression of at least one of the three critical molecules. One of these patients had reduced expression of both NY-ESO-1 and HLA-class I heavy chain while another had reduced expression of all three. A patient from the dose D group (NY-ESO-1 alone) was analyzed and showed reduced expression of NY-ESO-1 in the relapsing tumor. Immunogenic composition induced immune responses to NY-ESO-1 were observed in all five of the patients. The vaccine induced NY-ESO-1 specific immunity in the patients may have resulted in an immunological selection pressure resulting in the loss of antigen expression and or presentation observed in the relapsing tumors. Finally, one additional placebo patient was analyzed which indicated no change in expression of the three critical molecules in the relapsing tumor.

The foregoing examples demonstrate several features of the invention, which relates to methods for treating or preventing cancer in subjects who express the antigen referred to as NY-ESO-1, by administering to the subject a formulation of NY-ESO-1 and a saponin based adjuvant, especially ISCOM.

The treatment is effective against any cancer where expression of NY-ESO-1 has been shown. Expression can be shown via, e.g., RT-PCR, immunological analysis of patient samples, such as serum, blood, urine, etc., analysis of T cells, both CD4⁺ and CD8⁺ specific to complexes of NY-ESO-1 derived peptides and MHC molecules, and so forth. As these methodologies are well known, it is routine to determine the expression of the NY-ESO-1 molecule.

Prophylactic methods as well as therapeutic methods are contemplated because, as the art shows, the expression of NY-ESO-1 is associated with cancer only. While expression in testis has been noted, it is well known that testis cells do not express MHC molecules, and as such are not targets for immune reactive cells. As such, if a subject displays NY-ESO-1 expression in some way, but tumors cannot be identified treatment in the matter described herein may be indicated in order to prevent onset of cancer.

The combination of NY-ESO-1 protein and ISCOM was effective in inducing a combined cellular and humoral response to NY-ESO-1. Both known, and previously unknown T cell responses were identified, in the context of both MHC Class I and Class II responses. The addition of the ISCOM adjuvant generated much stronger responses than did the use of protein alone.

In practice, the invention involves the administration of an effective amount of NY-ESO-1 protein, as defined infra, in combination with a saponin containing adjuvant to a subject in need thereof, who expresses NY-ESO-1. The mode of administration may vary. In the experiments described herein, subject patients received intramuscular injections. Other possible forms of administration include intravenous, oral, intradermal, sublingual, subcutaneous administration via suppository, nasal spray, timed releases patch, internal slow release device, and so forth. Other forms of administration will also be clear to the skilled artisan, and need not be reiterated here.

The amount of formulation administered will vary, based upon a number of factors, such as the severity of the condition, the overall health of the subject patient, as well as age, and so forth. In general, however, a dose of from about 10 to about 500 μg of protein in combination with about 10 to about 500 μg of saponin based adjuvant, more preferably, from about 25 to about 250 μg of each, even more preferably, from 50 to about 150 μg of each, and most preferably about 100 μg of each. While the examples supra used identical amounts of both the protein and the adjuvant, it is to be understood that this is not a requirement for the invention in its broadest sense.

“Protein” as used herein refers to all forms of the NY-ESO-1 protein, including, but not being limited to, the protein disclosed in SEQ ID NO: 8 of U.S. Pat. No. 6,525,177, cited supra, as well as the forms described in this patent at, e.g., example 9, consisting of amino acids 10-180 and 10-121 of SEQ ID NO: 8. Indeed, any fragment of NY-ESO-1 is to be considered a part of the definition of protein used herein. Fragment refers to any portion of the full-length NY-ESO-1 molecule which is large enough to be processed intracellularly, into a peptide which then forms a complex with an MHC molecule, be it MHC Class I or Class II, such as those fragments described by Gnjatic, et al., J. Immunol., 170:1191-1196 (2003), incorporated by reference. Also a part of this definition are synthetic, polytopes which contain a plurality of amino acid sequences found in NY-ESO-1, which are concatenated to each other in such a way that, when processed intracellularly, they form individual peptides which then complex with MHC molecules as described.

Also a part of the definition are homologues of molecules which correspond to amino acid sequences that are found in SEQ ID NO: 8 of the '177 patent. It is known that variations within the sequence of NY-ESO-1 amino acids may not impact their binding ability, and may in fact improve it. See, e.g., U.S. Pat. Nos. 6,417,165 and 6,605,711, incorporated by reference which show this. “Homology” as used herein thus refers to molecules which are at least 70% identical, preferably 80% identical and most preferably 90% identical, to all or a part of the amino acid sequence of NY-ESO-1 referred to herein, as long as they contain at least one amino acid sequence which corresponds to an MHC Class I or MHC Class II binder.

Also part of the invention is the homologous protein antigen LAGE (PCT Application No. WO 98 32855), which contains many immunogenic peptides shared with NY-ESO-1, combined with saponin adjuvants. Also part of the invention are combinations of NY-ESO-1 or LAGE with saponin based adjuvants such as ISCOM, together with additional TRAP antigens such as CT-antigens MAGEA1-A12, MAGE-C1/CT7, MAGE-CT/CT10, SSX-2, SSX-4, SSX-5. and differentiation antigens such as Melan-A, gp100, tyrosinase, NY-CO-58, NY-BR-1. These are exemplary but not exhaustive.

Also, claimed as part of the invention are the novel antigenic peptides and corresponding nucleic acid molecules which represent epitopes for NY-ESO-1 specific CD8⁺ and CD4⁺ T cells described herein. Method for using such claimed novel NY-ESO-1 peptides are described/disclosed in, e.g., patent applications WO 98 14468; WO 99 53938; WO 01 364531; WO 02 26778; and WO 02 068800, all of which are incorporated by reference or techniques which are otherwise known to the skilled artisan.

The invention also encompasses the administration of proteins, in accordance with the definition set forth herein, together with one or more immunoreactive, NY-ESO-1 peptides, together with the adjuvant. Many such peptides are known.

Other features of the invention will be known to the skilled artisan, and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

1.-33. (canceled)

34. A method for treating a subject suffering from a cancer, cells of which express NY-ESO-1, comprising administering to said subject an amount of a composition containing NY-ESO-1 protein and a saponin-containing adjuvant sufficient to induce an antibody response to NY-ESO-1 in said subject.

35. The method of claim 34, wherein the amount of said compositions is sufficient to induce both a CD4⁺ and a CD8⁺ T cell response.

36. The method of claim 34, comprising administering said composition intramuscularly or subcutaneously.

37. The method of claim 34, comprising administering said composition intradermally.

38. The method of claim 34, wherein said saponin-containing adjuvant comprises saponin and sterol.

39. The method of claim 38, wherein said NY-ESO-1 protein is associated via hydrophobic interactions with said saponin and sterol.

40. The method of claim 38, wherein said NY-ESO-1 protein is not associated via hydrophobic interactions with said saponin and sterol.

41. The method of claim 34, wherein said saponin-containing adjuvant is an ISCOM or ISCOMATRIX adjuvant.

42. The method of claim 34, wherein said composition comprises equal amounts of NY-ESO-1 and saponin-containing adjuvant.

43. The method of claim 34, comprising administering from about 10 to about 500 μg of NY-ESO-1 protein to subject.

44. The method of claim 34, comprising administering about 100 μg of NY-ESO-1 protein to said subject.

45. The method of claim 34, wherein the NY-ESO-1 protein comprises full-length NY-ESO-1 protein.

46. The method of claim 34, wherein the NY-ESO-1 protein comprises the amino acid sequence of SEQ ID NO:1.

47. The method of claim 34, wherein said subject is affected with a tumor.

48. The method of claim 34, comprising administering an amount of said composition sufficient to prevent relapse in said subject.
